(19) United States
(12) Patent Application Publication
(10) Pub. No.: US 2024/0090953 A1
(43) Pub. Date: Mar. 21, 2024

(54) TUMOR SENSOR SYSTEM FOR TUMOR LOCALIZATION, THERAPY RESPONSE, AND IMAGE GUIDED SURGERY

(71) Applicant: STICHTING HET NEDERLANDS KANKER INSTITUUT - ANTONI VAN LEEUWENHOEK ZIEKENHUIS, Amsterdam (NL)

(72) Inventors: Theodoor Jacques Marie RUERS, Amsterdam (NL); Ray BURKE, Amsterdam (NL)

(73) Assignee: STICHTING HET NEDERLANDS KANKER INSTITUUT - ANTONI VAN LEEUWENHOEK ZIEKENHUIS, Amsterdam (NL)

(21) Appl. No.: 18/260,948

(22) PCT Filed: Jan. 14, 2022
(86) PCT No.: PCT/EP2022/050756

§ 371 Date: Jul. 11, 2023

(30) Foreign Application Priority Data
Jan. 15, 2021 (NL) 2027323

(51) Int. Cl. A61B 34/20; A61B 5/06

(52) U.S. Cl. CPC A61B 34/20; A61B 5/062; A61B 2034/2051; A61B 2503/40; A61B 2560/0219; H02J 50/001

(57) ABSTRACT

A tumor sensor system having an implantable probe and an auxiliary device, the probe having at least one sensor configured to generate an electric signal in response to a physical stimulus, the at least one sensor having a position sensor configured to sense a signal from a position tracking system, the sensed signal indicative of a position of the probe in a space, the position sensor having at least one electromagnetic sensor configured to cooperate with an external electromagnetic field generator, and the at least one electromagnetic sensor is configured to detect a property of the electromagnetic field and send it as an electric signal to the processing circuit, at least one antenna for data communication by means of inductive communication, an energy harvesting coil, and transmitting the data via the at least one antenna to the auxiliary device.

### FIELD OF THE INVENTION

The invention relates to a tumor sensor system.

### BACKGROUND OF THE INVENTION

For many tumors, multiple treatment options are available. The most common treatment options are radiotherapy, chemotherapy, and surgery, in any suitable combination. Other treatment options may also be available, depending on the situation. In general it can be difficult to localize a tumor and/or to assess the response of the tumor to the treatment.

In relation to radiation therapy and surgery, it may be difficult to locate the tumor inside the body with sufficient accuracy. Surgical navigation systems are generally only applied for targets in rigid areas. For non-rigid areas, real-time tumor tracking can be included to compensate for anatomical changes, physiological tissue movement or tissue movement during surgery.

“Accuracy assessment of target tracking using two 5-degrees-of-freedom wireless transponders”, by R. Eppenga, K. Kuhlmann, T. Ruers and J. Nijkamp, International Journal of computer Assisted Radiology and Surgery (2020) 15:369-377, discloses a clinically cleared system using a wireless electromagnetic tracking technique designed for radiotherapy. It is limited to tracking maximally three wireless 5-degrees-of-freedom (DOF) transponders, all used for tumor tracking.

WO 2017/086789 A1 discloses a method and system for providing visual information about a tumor location in human or animal body, wherein an electromagnetic tumor sensor can be provided in the tumor and tracked to determine its location in space, which is mapped to a tumor model. A surgical tool sensor can be provided on a surgical tool, and tracked to determine its location in space, which is mapped to a surgical tool model. The body can be scanned to obtain information about an anatomical structure. A reference sensor can be provided on the body, and tracked to determine its location in space, which is mapped to the anatomical structure. A virtual image can be displayed showing the tumor model, located with the at least one tumor sensor, in spatial relationship to the surgical tool model, located with the at least one surgical tool sensor, and the anatomical structure, located with the at least one reference sensor.

“Expanding the use of real-time electromagnetic tracking in radiation oncology”, by A. P. Shah, P. Kupelian, T. R. Willoghby, and S. L. Meeks, Journal of Applied Clinical Medical Physics, Volume 12, Number 4, Fall 2011, discloses a 4D localization system (Calypso System®) that utilizes radiofrequency (RF) for wireless tracking during radiation therapy. Three small (8 mm length×2 mm diameter) beacon transponders are implanted in or near the target. Each electronic transponder consists of an AC electromagnetic resonance circuit encapsulated in glass. Localization of the transponders is achieved using an electromagnetic array consisting of four radiofrequency signaling coils and 32 receiving coils. RF signals are emitted from the array at selected pulse rates and are used to excite the transponders at their individually unique resonant frequencies. The transponders absorb some of the radiofrequency energy and re-emit that energy in the form of a decaying signal that is detected by the electromagnetic array. The transponder position is then detected relative to the array which, in mm, is calibrated to the room coordinate reference system by three rigidly-mounted infrared cameras. A misalignment of the target is detected by identifying shifts of the target from its prescribed location anytime throughout the treatment.

### SUMMARY OF THE INVENTION

It is an object of the invention to provide improved equipment to aid the treatment of a tumor.

A first aspect of the invention provides a tumor sensor system comprising an implantable probe, the probe comprising
at least one sensor configured to generate an electric signal in response to a physical stimulus;
at least one antenna for data communication; and
a processing circuit for generating data based on the signal generated by the sensor and transmitting the data via the antenna.

By means of the implantable probe, the sensor can perform measurements inside or very close to a tumor. This allows to track a tumor in space and the progress of the treatment of the tumor. It allows to detect changing properties of the tumor without having to rely on imaging modalities. Therefore, changes relevant for tumor diagnostics and/or treatment can be tracked more frequently and more easily than before. The processing circuit provides for digital communication of the measurement results, which allows for more seamless transmission.

The probe may further comprise an energy harvesting coil for harvesting energy from an electromagnetic signal. This feature allows the probe to be energized even after having been implanted.

The energy harvesting coil of the probe may be among the at least one antenna. For example, a particular antenna of the least one antenna of the probe may be the energy harvesting coil of the probe. This way, the energy harvesting coil may be advantageously used to receive and/or transmit a communication signal.

A certain antenna of the at least one antenna of the probe may comprise a coil configured to communicate by means of at least one of near-field communication, far-field communication, magnetic resonance, or inductance. The probe may need fewer or less complex components to be able to perform communication using such a technique, allowing further miniaturization. Also, it may provide for low energy transmissions with a nearby device.

The probe may comprise a position sensor configured to sense a signal from a position tracking system to detect a position of the probe in a space. This provides a smart implantable position sensor. By detecting the signal from the position tracking system in the probe and converting the signal into data inside the probe and transmitting the data by the probe, an improved position tracking system is provided. The implantable probe may interfere less with other wirelessly communicating devices in the vicinity, in particular other position sensors, thereby removing limitations on the number of position sensors used. The need for an external sensor array may be obviated.

The position sensor may comprise an electromagnetic sensor configured to cooperate with an external electromagnetic field generator, wherein the electromagnetic sensor is configured to detect a property of the electromagnetic field and send it as an electric signal to the processing circuit. Based on the measured property, it is possible to find out a position of the probe.

The position sensor of the probe may comprise at least two electromagnetic sensors, which are oriented in at least two angularly distinct directions. This provides information to determine the position more accurately and/or more efficiently.

The sensor of the probe may comprise at least one of: an ionizing radiation dose detector; a peak ionizing radiation dose detector; and/or a cumulative ionizing radiation dose detector. The dose detector provides a highly local detection of the dose. Thereby, radiation therapy may be monitored or controlled. Moreover, radiation safety may be monitored.

The sensor of the probe may comprise at least one of: a sensor to detect a property of a tissue surrounding the probe; at least two electrodes to detect an impedance of a tissue external to the probe; and/or at least one photodetector with optional light emitters to perform a measurement for optical tissue characterization. The implanted probe is capable to perform a measurement inside or very close to the relevant tumor tissue. Thereby, more accurate information about the composition of the tissue may be generated.

The probe may comprise a rigid circuit and a flex circuit following a circumference of the probe, wherein the components of the probe are arranged on the rigid circuit and the flex circuit. The flex circuit allows for an extremely compact design, because the components are arranged three-dimensionally rather than on a two-dimensional PCB.

The system may comprise a wireless power supply coil external to the probe, wherein the wireless power supply coil is configured to transmit an energy-containing electromagnetic signal to the probe. This allows to provide power to the probe after it has been implanted.

For example, a length of the probe is at most 2 centimeters, and/or wherein a width of the probe is at most 3 millimeters. Such dimensions may allow to implant the probe through a needle.

Another aspect of the invention provides an auxiliary device, the auxiliary device comprising an antenna configured to receive the data from the probe; and a transmitter configured to transmit data based on the received data to a host. The auxiliary device allows the probe to send the data using low-energy transmissions, because the auxiliary device may be positioned close to the probe.

The auxiliary device may comprise a coil for wirelessly supplying power to the probe by transmitting an energy-containing electromagnetic signal to the probe. This feature cooperates with the energy harvesting coil of the probe, so that the probe is powered.

The auxiliary device may be a handheld device with a housing encapsulating the antenna of the auxiliary device and the transmitter of the auxiliary device. This allows the auxiliary device to be easily handled. For example, the auxiliary device may be easily moved close to the probe when needed.

The auxiliary device may comprise a wired power supply. This provides a reliable source of power. The wire may also be used for communication with the host device.

The auxiliary device may comprise fixation means for fixing the auxiliary device to a patient or to a surgical patient support. This facilitates use of the auxiliary device and probe during a procedure, for example a surgical procedure or a radiotherapy session. This may further facilitate the use of the auxiliary device and probe at home, if the auxiliary device is attached to the patient. Such a design for the use at home would be useful for therapy response measurements. An example of fixation means is an adhesive layer.

The auxiliary device may be suitable for being temporarily placed in a human or animal living being during surgery. In case it is necessary to bring the auxiliary device even closer to the probe, it may be placed inside the body during a treatment.

The auxiliary device may comprise a surgical instrument, such as a hand-held surgical instrument. For example, the antenna and transmitter of the auxiliary device may be built into a surgical instrument that can be used during surgery. This way the auxiliary device can be brought close to the probe without interfering with the procedure.

The surgical instrument may further comprise a position sensor. This way, the position of the surgical instrument can be determined relative to a medical image and/or relative to the probe.

The system may further comprise a robot arm configured to hold and move the auxiliary device during surgery. This facilitates the use of the probe during robotic treatments.

The probe and/or the auxiliary device may be detectable by X-ray, ultrasound, or MRI. This may facilitate registration between the probe, the auxiliary device, and X-ray or ultrasound data or MRI data.

The system may further comprise a feedback control loop, wherein at least one of: a power emitted by the coil for transmitting the energy-containing EM signal from the auxiliary device to the probe depends on a signal sent from the probe to the auxiliary device; or the electromagnetic field generated by the EM field generator of the navigation system depends on a signal sent from the probe to the auxiliary device; or the processing circuit of the probe is configured to set an operating parameter of the probe based on a signal transmitted from the auxiliary device to the probe. The processing circuit of the probe may be able to generate and transmit feedback signals relating to e.g. the energy consumption and/or certain properties of the EM signals. These signals may be processed by a processor, which may be integrated in the auxiliary device or in a host device, and used to optimize control of the system, in particular the power supply coil and/or EM field generator and/or operation of the probe.

According to another aspect of the invention, a method is provided to be performed by an implantable probe in a tumor sensor system, the method comprising
generating an electric signal in response to a physical stimulus by at least one sensor;
generating, by a processing circuit, data based on the signal generated by the sensor; and
transmitting the data via an antenna.

According to another aspect of the invention, a method is provided to be performed by an auxiliary device in a tumor sensor system, the method comprising
receiving data from an implantable probe by an antenna; and
transmitting data based on the received data by a transmitter to a host.

The person skilled in the art will understand that the features described above may be combined in any way deemed useful. Moreover, modifications and variations described in respect of the system may likewise be applied to the method, and modifications and variations described in respect of the method may likewise be applied to the system.

### BRIEF DESCRIPTION OF THE DRAWINGS

In the following, aspects of the invention will be elucidated by means of examples, with reference to the drawings. The drawings are diagrammatic and may not be drawn to scale. Throughout the drawings, similar items may be marked with the same reference numerals.

FIG. 1 shows a block diagram of an implantable probe.

FIG. 2 shows an electrical block diagram of the probe.

FIG. 3 shows an example of an auxiliary device that can be attached to a patient.

FIG. 4 shows an example of a hand-held auxiliary device.

FIG. 5 shows an example of an auxiliary device with integrated coils.

FIG. 6 shows an electrical block diagram of the auxiliary device.

FIG. 7 shows a perspective view of a probe.

FIG. 8 shows a perspective view of a PCB the probe.

FIG. 9 shows a sketch of a tumor sensor system in use.

FIG. 10 shows a flowchart illustrating a method performed by an implantable probe.

FIG. 11 shows a flowchart illustrating a method performed by an auxiliary device.

FIG. 12 shows a flowchart illustrating a method of operating a tumor sensor system.

FIG. 13 shows another sketch of a tumor sensor system in use.

FIG. 14 shows a probe with an optical detection feature.

### DETAILED DESCRIPTION OF EMBODIMENTS

Certain exemplary embodiments will be described in greater detail, with reference to the accompanying drawings. The matters disclosed in the description, such as detailed construction and elements, are provided to assist in a comprehensive understanding of the exemplary embodiments. Accordingly, it is apparent that the exemplary embodiments can be carried out without those specifically defined matters. Also, well-known operations or structures are not described in detail, since they would obscure the description with unnecessary detail.

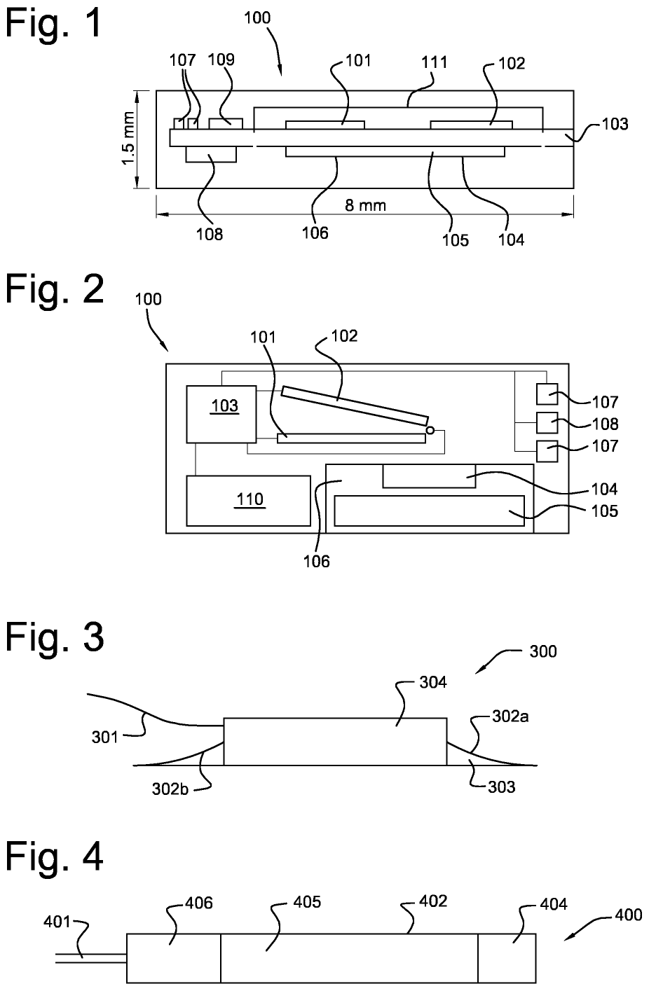

FIG. 1 shows a block diagram of an implantable probe 100. FIG. 2 shows an electric block diagram of the probe 100, illustrating how the components may be electrically connected.

In the shown embodiment, the probe 100 has a cylindrical shape with a diameter of 1.5 millimeters and a length of 8 millimeters. In a group of embodiments, the probe 100 is cylindrical with an outer diameter of at most 3 millimeters and a central axis of at most 20 millimeters. However, this is not a limitation. Other shapes are possible, such as rectangular or polygonal shapes. Advantageously, the length of the probe 100 is at most 20 millimeters and the width of the probe 100 is at most 2 millimeters. Even more advantageously, the length of the probe 100 is at most 15 millimeters and the width of the probe 100 is at most 2 millimeters. The above dimensions are provided for purpose of illustration. Other dimensions may be used alternatively. For example, the implantable probe 100 can be implanted by transporting the probe through the lumen of a needle into the tissue of a patient. A possible lumen size of a suitable needle is 9 gauge, which corresponds to approximately 2.91 millimeters, so that the width of the probe to fit through a 9 gauge needle would be less than approximately 2.91 millimeters. Other suitable needle sizes include, for example, a 16 gauge needle (corresponding to about 1.3 millimeters lumen diameter), and any size in between 9 gauge and 16 gauge. However, even smaller probes and thinner needles may be advantageously provided.

Advantageously, the probe has a smooth surface without sharp edges. In certain embodiments, the probe advantageously comprises an anchor (not illustrated) extending from the probe 100 to prevent drift of the probe 100.

For example, the implantable probe may be implanted in or near a tumor tissue. In certain embodiments (not illustrated) the edges may be rounded to improve the biocompatibility. Moreover, the outside of the implantable probe 100 may be made of a biocompatible material. In certain embodiments, this material is radiopaque, so that it may be visualized in a medical image, such as a CT scan. In certain embodiments, the material of the implantable probe may be detectable on a particular imaging modality, such as ultrasound, CT or MRI, so that its location within the anatomy of the patient may be determined after implantation. In certain other embodiments, the material of the probe may be selected to have a minimal disturbance of certain imaging modalities, such as MRI.

In certain embodiments, the probe and/or the auxiliary device may be MRI compatible, meaning that it can be imaged safely using MRI. Further, the MRI image is not significantly disturbed by the MRI-compatible probe, meaning that the position of the probe in relation to the tumor or healthy structure can still be determined.

In the embodiment illustrated in FIG. 1, the probe may be cylindrical or have shape resembling a grain of rice. However, this is not a limitation. For example, the probe may be equipped with anchors or other structures that facilitate fixation of the probe in the tissue. In alternative embodiments, the probe may have any different shape, for example a coiled shape. Initially, when the probe is introduced through a needle, the probe is straight, but when the probe is released from the needle into the tissue, the probe coils automatically. This may have the advantage that the probe can be longer in length.

The probe 100 comprises a processing circuit 103, for example an integrated circuit (IC). The processing circuit 103 is configured to control the electric components of the probe 100 and to process signals. In certain embodiments, this processing circuit 103 is implemented as an application specific integrated circuit (ASIC). An implementation in form of an ASIC has the advantage that it can be realized on a small footprint, so that the probe may be smaller in size. In certain embodiments, the processing circuit 103 may comprise an FPGA and/or a central processing unit (processor), and a memory with instructions, wherein the instructions cause the processor and/or FPGA to perform certain configured functions described herein, such as processing a signal from a sensor and transmitting a digital message indicative of the signal from the sensor.

The implantable probe 100 further comprises an antenna 104 configured to perform low-power communication with a nearby device. For example, the antenna 104 may use low-power Bluetooth.

Advantageously, the probe 100 may be configured to communicate by means of inductive coupling with the auxiliary device. For example, the auxiliary device may be held closely to the probe so that the antenna 104 of the probe 100 and the antenna of the auxiliary device 600 are within a wavelength distance apart. Alternatively, the probe 100 and the auxiliary device 600 may be configured to communicate with each other by means of electromagnetic wave radiation in the far field. This allows the communication to succeed if the antenna of the auxiliary device is more than a wavelength away from the antenna of the probe.

The antenna 104 may be configured to communicate via inductive communication. Examples of inductive communication systems include Near Field Communication (NFC), Far Field Communication (FFC), and magnetic resonance. To support an inductive communication system, the antenna 104 may comprise a coil. However, other low-power communication techniques are not excluded.

The electric components to generate the signals to be transmitted and to process received signals may be included in the processing circuit 103.

The probe 100 may comprise an energy storage device, such as a capacitor or a battery. In certain embodiments, the battery may be a non-rechargeable battery. In certain other embodiments, the battery may be a rechargeable battery. A rechargeable energy storage device, such as a capacitor or a rechargeable battery, may be charged using an energy harvesting component.

A particularly suitable energy harvesting component is an energy harvesting coil 105. Such an energy harvesting coil 105 is known by itself from, for example, wireless chargers. The energy harvesting coil 105 may cooperate with a wireless power supply coil 604. Such a wireless power supply coil 604 is configured to transmit an energy-containing electromagnetic signal to the probe. Advantageously, this wireless power supply coil 604 is included in the auxiliary device. However, in certain embodiments, the auxiliary device may comprise two separate (possibly handheld) devices: a wireless power supply device and a communication device.

In certain embodiments, the antenna 104 and the energy harvesting coil 105 are combined in a single coil 106. This allows for a compact design.

It will be understood that, for reasons of a compact design, the antenna 104 and the energy harvesting coil 105 may be implemented as a single coil 106 that combines both capabilities. In certain embodiments, the antenna 104 and the energy harvesting coil 105 may be implemented as separate components of the probe, for example as two separate coils. Moreover, the energy harvesting coil 105 is an optional feature.

In certain embodiments, the energy harvesting coil 105 may be configured to act as a reception antenna. Moreover, antenna 104 may be configured as a transmission antenna. This combination provides the advantage that the wireless power supply signal can be used to transmit communication signals from e.g. the auxiliary device 600 to the probe 100 efficiently, for example by modulating the wireless power supply signal, whereas communication signals from the probe 100 to e.g. the auxiliary device 600 can be more efficiently transmitted from an antenna 104 that is separate from the energy harvesting coil. For example, the energy harvesting coil 105, may be configured to receive communication signals by inductive telemetry, whereas the transmission antenna 104 may be configured to transmit communication signals by radiofrequency electric field telemetry (RF E field telemetry). In this case, electric field antenna 104 may comprise a monopole or dipole antenna, which may be implemented as a circuit trace on the flex PCB or rigid PCB. The processing circuit 103 of the probe 100 may further comprise a transmitter electric circuit electrically connected to the monopole or dipole antenna. In certain embodiments, the probe 100 is configured to be powered and receive communication from the external system (e.g. the auxiliary device 600) through the energy harvesting coil 105, and to transmit the generated data (e.g. position data and sensor data) through a separate antenna 104, which may be designed for electric field (E-field).

As illustrated in FIG. 2, the implantable probe may further comprise a power management module 110, to control energy harvesting, energy storage, and/or energy consumption.

The probe comprises at least one sensor to generate an electric signal in response to a physical stimulus. Examples of a sensor include position sensor 111, photodetector 180, and dose detector 109. The electric signal generated by the sensor may be processed by the processing circuit 103. The processing circuit 103 may comprise a digital-to-analog converter to digitize the signals received from the at least one sensor. The processing circuit 103 may further be configured to process the signals. Such processing may include, but is not limited to, for example, analyzing the signals, compressing the signals, and generating a control signal for controlling one of the components of the probe, such as the at least one sensor, based on the signals. Further, the processing circuit 103 may be configured to generate data based on the received signal, and transmit the data using the antenna. To this end, the processing circuit may further comprise a transceiver to generate and/or receive signals to be transmitted by and/or received by the antenna. In the following, several specific examples of relevant types of sensors are disclosed. These examples serve to illustrate rather than limit the present disclosure.

In certain embodiments, the probe 100 comprises a position sensor 111. The position sensor 111 is configured to sense a signal from an external position tracking system, so that the position in space of the probe may be determined using the sensed signal. Since the probe is implantable, the position sensor does not need a line of sight between the position sensor and the external position tracking system. The position sensor may be configured to sense the position including the orientation of the probe. For example, an electromagnetic position tracking system is used. In certain embodiments, the position sensor comprises at least one electromagnetic sensor 101 to sense an electromagnetic field.

In certain embodiments, the position sensor may be configured to detect the position with 5 degrees of freedom. In such a case, for example, the location in three dimensions and two rotation angles may be detected. The third rotation may remain unknown with such a position sensor. In certain alternative embodiments, the position sensor may be configured to detect the position with 6 degrees of freedom. In that case, for example, the location may be detected in three dimensions and three rotation angles may be detected. In certain alternative embodiments, the position sensor may be configured to detect the position with 3 degrees of freedom. In such a case, the position sensor may detect only the three coordinates of the location, and the rotation may remain unknown.

Advantageously, as illustrated in FIG. 1, the position sensor 111 comprises a first electromagnetic sensor 101 and a second electromagnetic sensor 102. The measurement direction of the first electromagnetic sensor 101 and the second electromagnetic sensor 102 may be different, so that the electromagnetic field strength may be measured in two different directions. For example, the first EM sensor 101 may be orthogonal to the second EM sensor 102. In certain embodiments, the position sensor 111 comprises at least two EM sensors with a different orientation. In certain embodiments (not illustrated), the position sensor 111 comprises at least three EM sensors, oriented differently to span three dimensions. However, it is noted that it may already be possible to detect position in 6 degrees of freedom (x, y, z, roll, pitch, yaw) with only two differently oriented EM sensors 101, 102.

In certain alternative embodiments, the position sensor 111 may be implemented using a different measurement principle, for example using an antenna array built into in the probe, to detect a position in space.

In certain embodiments, the implantable probe 100 may comprise one or more different measurement tools. Such additional measurement tools may be used for monitoring treatment effects and/or hazards. Changes in tissue characteristics may be detected, for example by detecting changes in the optical response detected by the photodetector 108 before vs. after treatment by radiotherapy or chemotherapy. These measurement tools may be provided instead of, or in addition to the position sensor. In certain embodiments, the implantable probe comprises a sensor that senses a characteristic of a tissue near the probe. For example, in certain embodiments the implantable probe 100 may comprise a photodetector 108. The implantable probe may optionally further comprise an one or more light emitters, such as a light emitting diodes 107 or another kind of lamp, so that the photodetector 108 can detect the light of the light emitting diode(s) 107 after the light has interacted with the tissue. Preferably the light emitter is capable of sequentially emitting light in different frequencies. For example, a plurality of light emitters 107 is provided, each light emitter configured to emit light in a different frequency band. By detecting the diffracted light with the light detector 108, it may be possible to characterize the tissue around the implantable probe. In the example embodiment of FIG. 1, the implantable probe comprises two light emitting diodes 107. In certain other embodiments, the light may be generated by a luminescent marker or another light source external to the implantable probe. The photodetector may include optical filters, for example to allow to detect a specific wavelength range of light.

The light emitters 107 may be configured to emit light of at least two different wavelengths. This may facilitate SPO2 detection and/or other biomarker reflectance based measurements.

In certain embodiments, the probe may comprise at least two electrodes 810a, 810b, to detect an impedance of a tissue external to the probe. The impedance may be a valuable property to characterize tissue around the implantable probe. By including electrodes this property may be assessed and changes to this property may be detected. These electrodes may be exposed to the outside of the probe 702, through an opening in the wall 701. However, this is not a limitation. In certain embodiments the electrodes can perform electric measurements through the wall 701. The electrodes 810a, 810b may have a gold finish and may be part of the flexible PCB, which may comprise a top metal layer.

In certain embodiments, the implantable probe 100 further comprises a radiation dose detector 109, for example an X-ray dose detector, a gamma radiation dose detector, or a proton radiation dose detector. The dose detector 109 may comprise a peak dose detector 109a and/or a cumulative dose detector 109b. This feature may be useful, for example, to ascertain that an appropriate dose has been applied to the target around the implantable probe, during radiotherapy. The peak dose detector 109a may comprise a scintillator that creates, in response to ionizing radiation, photons in a wavelength spectrum that is detectable by a photodiode.

The cumulative dose detector 109b may comprise a FET-based radiation sensor. Such a sensor technique is known by itself, for example from Varadis RADFET™, which may comprise a discrete p-channel MOSFET optimized for radiation sensitivity. RADFETs are sensitive to ionizing radiation: gamma rays, X-rays, and/or protons. The RADFET comprises a gate oxide. Through generation and trapping of radiation-induced charges in the gate oxide, radiation exposure changes the output voltage of the RADFET and this change is related to the radiation dose. The read-out can be performed by forcing a DC current (in the range of 10s of μA) into the device and measuring the DC voltage (in the range of 0.5 to 8 Volts). The RADFET response may be non-linear and a pre-recorded calibration curve may be used to read the dose. This calibration curve may be applied, for example, by the processing circuit 103. Alternatively, the calibration curve may be applied by the auxiliary device or by a host device.

In certain embodiments, the implantable probe may further comprise at least one capacitor 809. The capacitor 809 can store energy, such as the energy harvested using the energy harvesting coil 105. This way a continuous energy supply may be realized.

The components of the implantable probe may be integrated on an ASIC, for example, or on at least one PCB, such as a rigid PCB. In certain embodiments, the components may be integrated on a flexible PCB. A flexible PCB may be a layer of material that can be bent. For example, the flexible PCB may be bent to form a cylinder by attaching two opposite edges to each other. In certain embodiments, a combination of a rigid PCB and a flexible PCB may be used. Two opposite edges of the flexible PCB may be attached to the rigid PCB, for example.

Referring to FIG. 7, the implantable probe may have a housing 701 with electric components inside. The housing 701 may be made of a biocompatible material and may have a generally cylindrical shape. However, other shapes may also be possible, such as a spherical shape or a rectangular shape or even other shapes. The electronics may be arranged on at least one board 702, which may be a printed circuit board (PCB). In certain embodiments, the board 702 has a rounded shape matching the cylindrical outline of the housing 701. In certain embodiments, the board 702 comprises a flexible board 802, which may be folded in such a rounded shape. The board 702 may further comprise a rigid board 801. In certain embodiments, as illustrated, two opposing edges of the flexible board 802 may be attached to the rigid board 801. For example, as illustrated, two opposing edges of the flexible board 802 may be attached to two opposing edges of the rigid board 801.

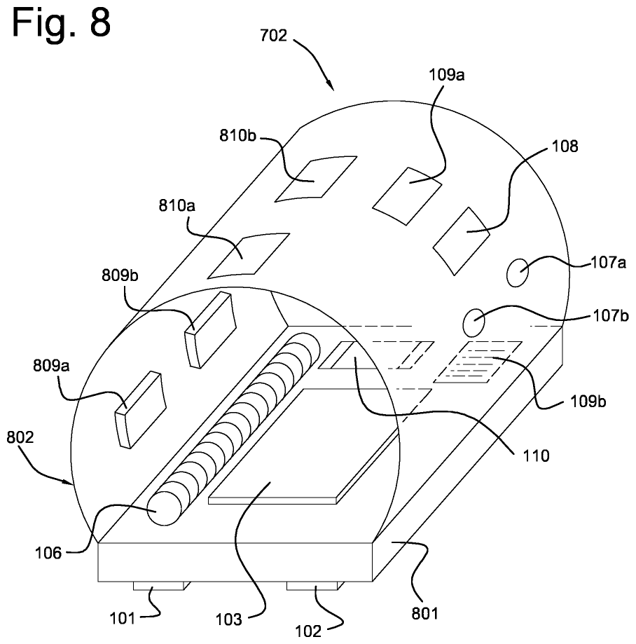

FIG. 8 shows a perspective view of the rigid board 801 and the flexible board 802 of FIG. 7, in greater detail. The components of the implantable probe have been illustrated as being mounted on the rigid board 801 or the flexible board 802. However, it will be understood that the components may be distributed differently. In certain embodiments, one of the rigid board 801 and the flexible board 802 may be omitted. In the illustrated example implementation, an integrated circuit 103 is mounted to the rigid board 801, as well as the coil 106 for communication and energy, a power management unit 110, and a cumulative dose detector 109b. The position sensor, implemented in this case as a first EM sensor 101 and a second EM sensor 102, may be mounted on the rigid board to better fixate their relative orientation. For example, the EM sensors 101, 102 may be mounted on the opposite side of the rigid board 801, facing the housing 701. Mounted to the folded flexible board 802 are, for example, the capacitor 809, the electrodes 810, a peak dose detector 109a, a photodetector 108 and one or more LEDs 107a, 107b. Other arrangements of the components are also possible.

FIG. 3 shows a first example of an auxiliary device 300. The auxiliary device 300 comprises a housing 304. The electric components inside the housing 304 of the auxiliary device 300 are not shown in FIG. 3, but they will be explained below with reference to FIG. 6. The auxiliary device 300 has a lead 301 to provide an electric connection to, for example, an external power supply and/or a wired communication line to a host device. The auxiliary device 300 further has attachment means 302a, 302b, to attach the auxiliary device 300 to the skin 303 of a patient. This way, by placing the auxiliary device 300 on the outside skin of the patient, the auxiliary device can be brought as close as possible to the implantable probe without being invasive. In certain embodiments, however, the auxiliary device 300 may be configured to be placed, and optionally fixated within, the human or animal body. For example, the auxiliary device 300 may be implemented as an endovascular, endobronchial, colonoscopy, or any endoluminal device or another catheter device, or may be fixated to tissue within the body. For example, the auxiliary device may comprise an adhesive surface to attach the auxiliary device to tissue inside the body, for example an organ such a as a liver or a kidney. In certain embodiments, the auxiliary device 300 may be configured to be fixed to a patient bed, in particular an interventional patient support, for example a surgical patient support or a patient support of a radiotherapy apparatus. In certain embodiments, the lead 301 may be omitted and replaced by an optional battery and wireless communication unit. In certain embodiments the auxiliary device may have a sensor (e.g. position sensor, dose detector, photodetector, or impedance detector), and the auxiliary device may communicate outputs of the sensor to the host.

FIG. 4 shows a second example of an auxiliary device 400. The auxiliary device comprises a housing 402. The electric components inside the housing 402 of the auxiliary device 400 are not shown in FIG. 4, but they will be explained below with reference to FIG. 6. The auxiliary device 400 has an optional lead 401 to provide an electric power connection and/or a wired communication connection. The auxiliary device of this second example is a hand-held device, which may be for example implemented as a pen-like, or elongated object. The auxiliary device 400 may be a medical tool, such as a surgical tool. For example, a pointer device or a surgical clip. In other words, the housing 400 of the auxiliary device may be the housing of a medical tool, which can be used during surgery by the surgeon, in the vicinity of the implantable probe. In certain embodiments, the components of the auxiliary device 400 that directly interact with the implantable probe 100, such as an antenna and/or a power supplying coil 604 for wireless power supply, may be positioned close to the distal tip 404 of the housing 400, so that they can be manually brought as close as possible to the implantable probe 100. The remaining electrical components may be positioned in the middle portion 405 or proximal portion 406 of the auxiliary device. The surgical tool may further comprise another position sensor, preferably located at the distal tip of the surgical tool.

In certain embodiments, the auxiliary device 400 is a hand-held tool that can be shaped as a “box” that can be put anywhere near the patient. Whether the auxiliary device is built into a surgical instrument or not, by way of illustrative example, the length of the auxiliary device may be less than 20 centimeters and the width and height may be less than 6 cm. Preferably, the length of the auxiliary device may be less than 5 centimeters and the width and height may be less than 2 centimeters each.

In certain embodiments, the auxiliary device is suitable for being implanted in a human or animal living being. For example, the auxiliary device is implemented as compact as possible and its outside housing is made of a biocompatible material. In certain embodiments, the auxiliary device is suitable for being placed in a human or animal living being during a surgical intervention, to be removed before the surgical intervention ends. Moreover, as in other embodiments, the auxiliary device may have an electric lead 401 for power supply and/or data communication with a host, or it may be implemented completely wirelessly with a battery and wireless communication with the host.

In certain embodiments, the auxiliary device may be held by a robot arm instead of a surgeon. Although this applies to any of the example auxiliary device 300, 400, 500, this may be particularly useful for the portable auxiliary device 400 so that it can automatically be positioned near the probe when needed.

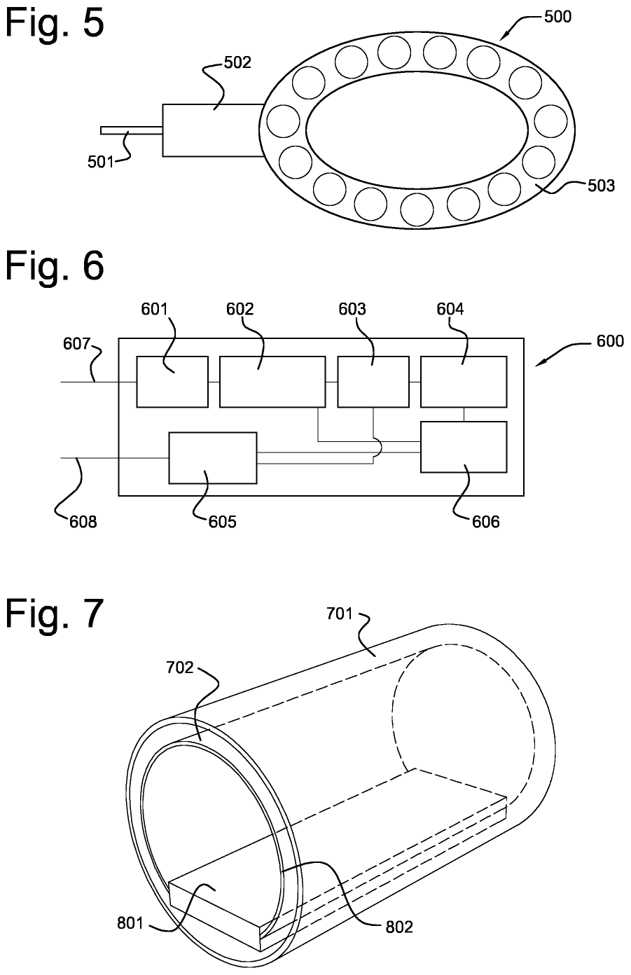

FIG. 5 illustrates an exemplary auxiliary device 500 with a first part 502 housing the electrical components. The auxiliary device 500 may further comprise an optional wire 501 for power supply and/or communications. The auxiliary device 500 may further comprise a plurality of navigation coils 503. The coils 503 may be used to generate a sequence of electromagnetic fields that can be detected by the EM sensor(s) 101 and 102 of the implantable probe. For example, each coil 503 may be activated in sequence to generate an electromagnetic field. The measurements made by the EM sensor(s) 101 in response to the generated sequence of electromagnetic fields can be used to calculate the position of the probe 100. Such calculations may be made by any of the host device, the auxiliary device 300-600, or by the probe 100.

Optionally the coils 503 may also be used to provide power to the implantable probe and/or to communicate with the implantable probe.

For example, the auxiliary device 500 of FIG. 5 may be placed underneath the patient or may be built into the patient support in an operating room. The electronics 502 of the auxiliary device then is still relatively close to the implantable probe.

In certain embodiments it may be preferred to provide a separate unit with the navigation coils 503. These coils may be placed at a suitable place, for example below the patient, while the auxiliary device 300, 400 may be put at another suitable place, for example nearby the probe 100.

FIG. 6 shows a simplified electric diagram of an example implementation of the auxiliary device 600. The diagram may be applied as an example electrical implementation of each of the examples 300, 400, and 500.

The auxiliary device 600 may comprise a first communications unit 601 comprising a transceiver for wired communication through lead 607 with a host device 901. Alternatively, the transceiver of the first communications unit 601 may be configured for wireless communication with the host device.

The auxiliary device further comprises a processing circuit 602, such as a digital signal processor (DSP) or a microprocessor, or an ASIC, to control operations of the auxiliary device 600.

The auxiliary device further comprises a second communications unit 603, i.e. a transceiver, to communicate wirelessly with the probe. The second communications unit 603 may comprise, for example, an NFC chip or a chip for far-field communication. The second communications unit 603 may comprise a transceiver that utilizes electromagnetic resonance in either or both of the transmission coil and reception coil. Alternatively, the second communications unit 603 may comprise any type of transceiver, corresponding to the type of transceiver of the probe 100, such as a low-energy Bluetooth transceiver with suitable antenna.

The auxiliary device 600 may further comprise a power supply unit 605 to regulate the power. The power supply unit 605 may comprise a battery and/or may provide a wired connection to an external power supply via a wire 608.

The auxiliary device 600 may further comprise one or more coils 604 and a coil controller 606. The one or more coils 604 may comprise a coil for wireless power supply to the implantable probe, a coil for data communication under control of the transceiver 603, and an optional navigation coil to generate an EM field for the position sensor of the implantable probe. It will be understood that a navigation coil may be provided as a separate object external to the auxiliary device. In certain embodiments, any of the coils may be combined in a single coil. For example, the coil for wireless power supply and the antenna coil for data communication may be combined in a single coil. In certain embodiments, one or more of the EM field generating coils may perform the function of the wireless power supply coil and/or data communication coil. In certain embodiments, the wireless power supply coil generates sufficient energy to enable operation of one or more probes.

In certain embodiments, the coil 604 for wireless power supply may be electrically connected to a transmission circuit of the communication unit 603. The coil 604 may be configured to modulate the wireless power supply signals generated by the coil 604 in order to transmit (e.g. data) communication signal to the probe 100. The communication unit 603 may further comprise a reception circuit comprising an antenna configured to receive (e.g. data) communication signal from the probe 100. For example, the reception circuit comprise an RF E-field antenna and may be configured to receive signals from the probe 100 using electric field (E-field) telemetry.

In certain embodiments, the RF E-field antennas of the probe 100 and auxiliary device 600 may be configured to perform bidirectional communication, whereas the power supply coil 604 and energy harvesting coil 105 may be configured to perform single-directional communication from the auxiliary device 600 to the probe 100.

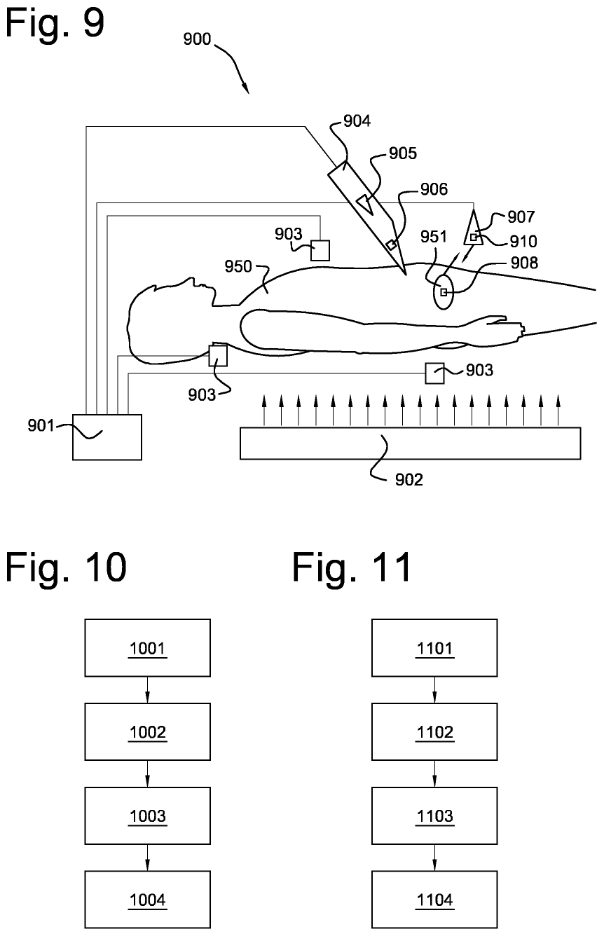

Referring to FIG. 9, in certain embodiments, the auxiliary device 907 comprises a position sensor 910, which may be implemented similar to the position sensor 101,102 of the probe 100. This position sensor 910 may detect the position of the auxiliary device 907 with respect to the patient 950 and/or the navigation system, in particular the EM generating coil 902. The auxiliary device may be configured to transmit the position detection result to the host.

It will be understood that the field generating coil(s) of the position tracking system may be separate from the auxiliary device. Alternatively, the auxiliary device may comprise certain components of the position tracking system such as a field generating coil. However, in case an electromagnetic tracking system is used, the coils for generating an appropriate electromagnetic field may be provided separately and may be mounted at appropriate positions according to the specifications of the position tracking system. For example, such coil or coils may be provided underneath the patient, in between the patient and the patient support, for example, or they may be built into the patient support. Alternatively, the components of the position tracking system may be provided elsewhere, for example above the patient.

In certain embodiments, one of the implantable probe and the auxiliary device, or both, may be detectable by X-ray or ultrasound or magnetic resonance imaging (MRI). This way, the position of these objects can be co-located with anatomical structures.

In certain embodiments, the auxiliary device comprises at least two separately movable units: one unit comprising the power supply coil and another unit comprising the antenna for data communication.

In certain embodiments, an active feedback control loop is implemented. Certain features of the implantable probe and/or the auxiliary device may be controlled, based on feedback acquired from the implantable probe. For example, the intensity of the power transmitted by the wireless power supplying coil of the auxiliary device may be adjusted based on a signal from the wireless probe, indicative of a need for power. This allows to adjust the power to the current circumstances, which may be different depending for example on the distance between the auxiliary device and the implantable probe and/or the tissues in between these devices and/or the distance to the components of the position tracking system. Also, the electromagnetic field generator of the position tracking system may be adjusted based on a feedback signal obtained from the implantable probe. Also, the sensors and the other components of the probe may be controlled by a command that is sent from the auxiliary device to the probe. For example, a measurement interval may be controlled, or a communication frequency may be set.

In certain embodiments, the resonance impedance of the coil 106 of the probe 100 and/or the coil 604 of the auxiliary device 600 may be actively controlled. In general if the energy harvesting coils and the antenna are maintained at resonance the energy transfer and the communications can be at their strongest and their best. By controlling this resonance, changes in environmental conditions or conditions relating to the patient, such as body-mass-index (BMI) or other medical conditions may be taken into account. Other conditions to take into account may include the position and orientation of the probe in relation to the position and orientation of the auxiliary device. Other conditions to take into account may include the internal electric properties of the components of the probe 100 and/or the auxiliary device 600, which may be slightly different in each individual device and which may even change during the lifetime of the device. By actively monitoring the resonance of the device and actively controlling the operation of the probe 100 and/or the auxiliary device 600 based on the detected resonance state, such variations may be addressed.

For example, the processing circuit 103 of the probe 100 may be configured to detect the resonance condition of the energy harvester coil 105 and/or the communication coil 104. For example, this may be implemented by evaluating the amount of energy received via the relevant coil. Based on the detected resonance condition, the processing circuit 103 may modify the impedance of the circuit actively. Such a modification of the impedance may be realized by controlling a switch that can selectively connect an additional capacitance or inductance in the circuit including the coil 106. For example the extra capacitance or inductance may be electrically connected in sequence or parallel to the coil 106. An alternative way is to vary the stimulation frequency at the system end via communication to come back into resonance. For example, the probe 100 may send a signal to the auxiliary device 600 to request a change of transmission frequency. In response, the auxiliary device 600 may change its transmission frequency accordingly. This active control facilitates the use of a relatively small coil and/or a coil made of MRI compatible material. For MRI compatibility, the use of soft magnetic materials may be undesirable. As a consequence of this, the quality factor (Q factor) may be relatively high. Because of these circumstances, the frequency bandwidth for resonance may be relatively narrow. The active monitoring and control of the resonance of the coil is advantageous in this respect because it provides reliable operation of the probe.

The tumor sensor system may comprise further position sensors. These position sensors may be implemented in the same way as the implantable probe 100. Alternatively, the position sensors may be wired position sensors so they may have a wired power supply and/or wired communication to a host device (or to the auxiliary device) instead of the wireless power coil and/or antenna. Such position sensors may be built into surgical tools, including the auxiliary device itself. Moreover, position sensors may be fixed on the outside (skin) of the patient, for example by means of an adhesive. Also, the other types of sensors may be implemented in a similar way to perform measurements on the skin. Also, position sensors or other types of sensors may be incorporated into a surgical tool.

Software may be used to visualize the tools, anatomical structures, and the tumor based on the positions sensed by the position sensors, and preoperative image data. The position tracking system may be calibrated by pointing a surgical tool with a position sensor in it to a known anatomical structure, determining the position of the surgical tool based on the position sensor, projecting the tool in the preoperative image using the determined position, and comparing the real position of the surgical tool to the projected position in the preoperative image.

Although the description has focused on the description of one probe and one auxiliary device, this is not a limitation. The system may comprise a plurality of implantable probes 100 that can operate simultaneously. One auxiliary device 600 may be used to operate a plurality of implantable probes 100. Depending on the geographic distribution of the probes 100, it may be necessary to move around the auxiliary device 600 towards at least one probe that is to be activated. Alternatively, multiple auxiliary devices 600 may be operated simultaneously, wherein each auxiliary device 600 may operate one or more probes 100.

Passive beacons transmit a signal that is received by a sensor array to detect the position of the beacon. Therefore, signals transmitted by beacons can interfere with each other, thus limiting the number of beacons that can be used simultaneously. In contrast, certain embodiments of the probes with position sensor as disclosed herein measure the electromagnetic field that is generated by the magnetic field generating navigation coils. The measurement value is transmitted to the auxiliary device using a communication protocol, so that the transmissions do not interfere. Moreover, the power supply to the probe is not realized by the navigation coils, but by the power supply coil in the auxiliary device. Therefore, the electromagnetic field of the navigation coils does not have to contain so much energy. Since the auxiliary device can be held more closely to the probe than the navigation coils, wherein the exact position of the auxiliary device plays no role in the position detection, the power can be provided more efficiently from the auxiliary device to the probe. More probes can be used for position detection simultaneously, using the same electromagnetic field generated by the navigation coils.

In certain embodiments, the navigation works by having field coils (‘navigation coils’) fixedly aligned in different axes. When the navigation coils are turned on, they energize the position sensor a particular way. The position sensor detects this and the processing circuit generates measurement data out of the signal generated by the position sensor. This measurement data is transmitted from the probe to the auxiliary device. Based on the measurement data, the spatial position and orientation of the probe can be calculated.

FIG. 9 illustrates a tumor sensor system 900 and a patient 950 with a tumor 951. The tumor sensor system 900 may comprise a host 901. The host 901 may act as a control unit of the tumor sensor system 900. The host 901 may comprise a computer, for example a medical workstation, with a microprocessor, memory, and suitable communication modules. Alternatively, the host 901 may be implemented with dedicated hardware.

Inside the tumor 951 is a wireless implantable probe 908. In general a wireless implantable probe may be implantable in any tissue. It is not limited to a tumor in this regard. For example, the implantable probe 908 may be implanted in a tissue adjacent to the tumor 951. Moreover, auxiliary device 907 with optional position sensor 910 is configured to facilitate wireless communication with the implantable probe 908 and/or provide a wireless power supply for the implantable probe 908. In a group of embodiments, the auxiliary device 907 is not an implantable device.

The tumor sensor system 900 may further comprise a navigation system to detect the positions of several components of the tumor sensor system 900. FIG. 9 illustrates the example of an EM based navigation system. To that end, the tumor sensor system 900 comprises an EM field generator 902. In certain embodiments, the EM field generator 902 is configured to generate several different electromagnetic fields in a sequence. The EM field generator 902 may comprise a plurality of coils that each generate a (slightly) different EM field. For example, the coils of the EM field generator may be positioned and/or oriented differently with respect to each other. By activating each coil in a sequence, a plurality of different EM fields is generated.

In certain embodiments, the sensor system further comprises one or more wired probes 903. These wired probes may be powered and/or controlled by the host 901 through a wire. Therefore, such probes are particularly suitable for measurements at well-accessible locations, such as the outside skin of a patient. Moreover, a wired probe 906 may be included in a wired surgical instrument 904. In certain embodiments, a probe on or in a surgical tool can be a wireless probe.

Each probe 903, 906, 908, and/or 910 may comprise a position sensor. In certain embodiments, each position sensor includes one or more EM sensors, which detect an electromagnetic field strength. These EM sensors may be directional, so that they detect an EM gradient in a particular 3D direction. In certain embodiments, a position sensor comprises at least two EM sensors, which have a different orientation. That way, the EM field strength can be measured in two different directions. The host 901 may control the EM field generator to generate a sequence of different EM fields. At the same time, the position sensors of the probes 903, 906, 908, and/or 910 may be configured to detect the EM field strengths corresponding to each generated EM field. This way, sufficient measurement data is generated that allows to calculate the positions of the probes 903, 906, 908 and/or 910.

In certain embodiments, at least one of the probes 903, 906, 908, 910 may comprise other sensors configured to perform other measurements, such as tissue characterization measurements. These other sensors may be provided instead of, or in addition to, the position detector.

FIG. 13 illustrates an alternative tumor sensor system 1300. In the figure, the same reference numerals are used as in FIG. 9 for the components of the tumor sensor system. These implementation of these components is similar to the components shown in FIG. 9 and the details thereof are not repeated here. In particular, the tumor sensor system 1300 comprises an implantable probe 908 (or a plurality of implantable probes), an auxiliary device 907, and a host 901. The probe 908 comprises at least one sensor as described above, which is not a position sensor. The auxiliary device 907 may be configured to transmit a command to the probe to perform a measurement using the sensor and transmit the result back to the auxiliary device. However, the EM field generator 902 is omitted in the example illustrated in FIG. 13, because no position detection is performed. Such a setup may be useful to detect e.g. response to a therapy by sensing a tissue characteristic, such as an optical characteristic of the tissue around an implanted probe. The probe 908 may receive power from the auxiliary device 907, in the way described hereinabove.

In certain embodiments, the host 901 may be remote from the auxiliary device 907. For example, the communication between the host and the auxiliary device may go through a wide area network connection, such as the Internet. The host 901 may be a cloud service or a server in a remote hospital.

It will be understood that the probes 903, 906, 910 and the auxiliary device 907 may communicate with the host 901 by means of wired communication or wireless communication. In case of wireless communication, any suitable communication technology may be employed, such as Bluetooth or Wi-Fi. The implantable probe 908 may be configured to communicate wirelessly with the auxiliary device 907, preferably using inductive coupling.

In certain embodiments, the tumor sensor system 900 further comprises at least one auxiliary device 907. The auxiliary device 907 typically does not need to have a fixed or known location, unless it includes the EM field generator. However, the auxiliary device 907 may facilitate the operation of at least one wireless probe 908. Thanks to this support function, the probe 908 may be further miniaturized.

The auxiliary device 907 may communicate with the wireless probe 908 by means of any communication technique. In particular, a communication technique may be implemented with a short range. For example, the supported distance between the auxiliary device 907 and the probe 908 may be, for example, 10 cm or less, or 20 cm or less, or 30 cm or less, depending on the application and the size of the patient 950.

In certain embodiments, magnetic resonance method for communication may be based on an amplitude modulation or a frequency modulation around the resonance mode of the coil or coils of the signal sent from the probe 100 to the auxiliary device 600 or vice versa. The carrier frequency of this signal may correspond to the resonance frequency of the energy harvesting and/or antenna coil 106 of the probe 100 and/or the corresponding coil 604 of the auxiliary device 600. The frequency and/or amplitude of this signal may be modulated in order to transmit a signal from the auxiliary device 600 to the probe 100. Similarly, the probe 100 may transmit a signal to the auxiliary device 600 by transmitting a signal at the resonance frequency and modulating it.

The auxiliary device 907 may be configured to control the probe 908 by sending commands to the probe 908. Alternatively, the probe 908 may operate independently. In both cases, the probe 908 sends measurement data and optional status information to the auxiliary device 907. The auxiliary device 907 may be connected to the host 901 by a wired connection or a wireless communication connection, such as Bluetooth or Wi-Fi. The auxiliary device 907 may act as a repeater and forward any communication from the host 901 to the probe 908 and forward any communication from the probe 908 to the host 901. In certain embodiments, the auxiliary device 907 performs data processing. For example, the auxiliary device 907 may generate frequent reports of the data received from the probe 908 and send the reports to the host 901.

In certain embodiments, the auxiliary device 907 may be configured to provide wireless power to at least one probe 908. This may be performed using e.g. Qi (TM of ‘The Wireless Power Consortium’) compatible wireless power supply, for example a loosely coupled, resonant wireless protocol of power supply.

In certain embodiments, an auxiliary device 905 may be incorporated into a surgical tool 904. This way, the auxiliary device 905 is close to the wireless probe 908 without being obstructive. In certain embodiments, multiple auxiliary devices 907, 905 may be supplied. For example, based on a measurement of connection quality between the wireless probe 908 and each of several operative auxiliary devices 907, 905, one auxiliary device may be chosen to take care of the wireless probe 908. For each wireless probe 908 in the system, the choice of auxiliary device may be different (or the same), depending on the connection quality. It will be understood that any surgical instrument may comprise either an auxiliary device 905 or a position sensor 906, or both.

In certain embodiments, at least one auxiliary device has the host 901 built-in. That is, in certain embodiments, at least one of the auxiliary devices can act as the host 901. In other words, the functionality of the host 901 and one of the auxiliary devices may be integrated in one physical unit. It is also possible that some of the functionality of the host 901 is implemented by the auxiliary device(s) 907, 905.

The host 901 may control the EM field generator 902 and/or may collect all the measurement data from the probes 903, 906, 908, 910 for processing and/or visualization of the results. For example, the host 901 has a calculating unit to perform the calculations that are necessary to calculate the position of each probe based on the measurements of the position sensor. However, such calculations may alternatively be performed, in part or in its entirety, by the auxiliary device 907, 905, or by the probe 903, 906, 908, 910 itself. The same applies to processing steps to be performed on the measurement data generated by the other possible sensors of the probes 903, 906, 908, 910, such as impedance measurements, optic measurements, temperature measurements, and any other measurements.

In certain embodiments, the operation of the tumor sensor system 900 comprises a feedback control loop. For example, the probes 903, 906, 908, 910 can generate feedback regarding the EM field strength, which may be used to adjust an operating parameter of the EM field generator. This feedback may be received from a wireless probe 908 by the auxiliary device 907, and forwarded to the host 901, which can determine an adjusted operating parameter for the EM field generator, and send a corresponding control signal to the EM field generator.

In certain embodiments, a feedback control loop may be implemented to optimize wireless power delivery to the wireless probe 908. For example, energy harvesting result may be determined by the wireless probe 908 in addition to an optional power demand value. These values may be transmitted to the auxiliary device 907, 905. In response, the auxiliary device 907, 905 may adjust the power transmission to the wireless probe 908. Alternatively, the auxiliary device 907, 905 or the host 901 may generate a guiding indicator for the user or the robot to move the auxiliary device 907, 905 to a more favorable position.

In certain embodiments, a feedback control loop may be implemented to optimize wireless data communication quality and/or efficiency between the probe 908 and the auxiliary device 905, 907. For example, in case of data reception errors, the transmitting device may be instructed by means of a wirelessly transmitted feedback signal, to increase transmission power. Alternatively, in case of connectivity problems, the auxiliary device 905, 907 or the host 901 may be configured to generate a guiding indicator for the user or the robot to move the auxiliary device 907, 905 to a more favorable position.

In certain embodiments, one of the implantable probe 100 and the auxiliary device 600, or both, may be detectable by X-ray or ultrasound or magnetic resonance imaging (MRI). For example, the material of these devices may comprise a radiopaque material.

FIG. 10 illustrates steps of a method that may be performed by an implantable probe in a tumor sensor system. In step 1001, the probe optionally generates electricity using an energy harvesting component, such as a wireless power receiver. In step 1002, the method starts by generating an electric signal in response to a physical stimulus by at least one sensor of the probe. In step 1003, a processing circuit of the probe generates data based on the signal generated by the sensor. This step may comprise analog-to-digital conversion by an analog-to-digital converter (ADC). Moreover, it may comprise compressing the data. In step 1004, a transmitter of the probe transmits the data via an antenna of the probe.

FIG. 11 illustrates steps of a method performed by an auxiliary device in a tumor sensor system. In optional step 1101, a wireless power transmitter of the auxiliary device transmits energy wirelessly to the probe. In step 1102, a receiver of the auxiliary device receives data from a probe, by an antenna of the auxiliary device. In step 1103, a control circuit of the auxiliary device processes the received data, and generates output data based on the received data. In step 1104, the transmitter of the auxiliary device transmits the output data to a host device.

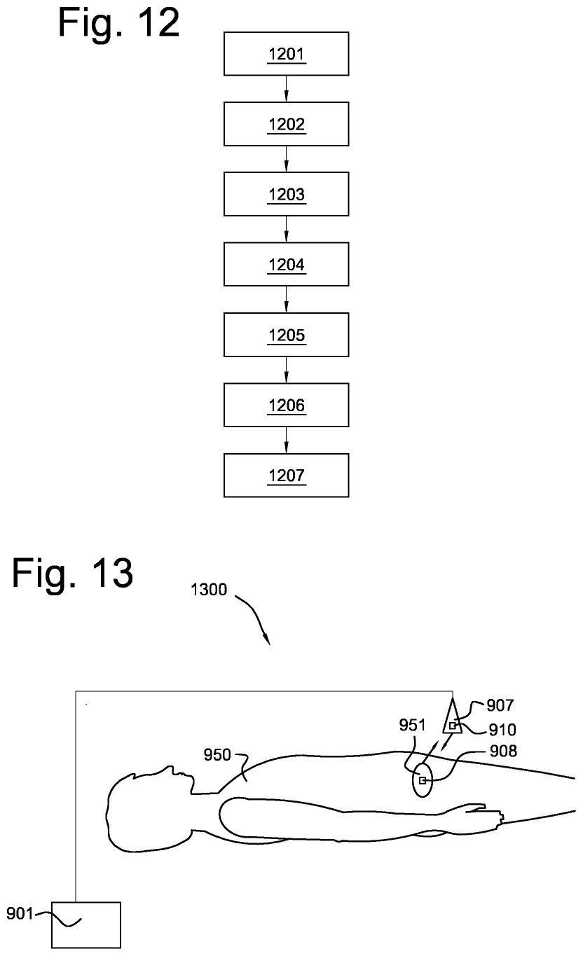

FIG. 12 illustrates steps of a method of operating a tumor sensor system, wherein the tumor sensor system comprises an implantable probe and an auxiliary device. The method starts at step 1201 by implanting a probe into a tissue, for example in a tumor tissue or close to a tumor tissue. In step 1202, the probe activates a sensor inside the probe to perform a measurement. After that, in step 1203, the probe generates data based on the measurement. For example, the data is indicative of a measured quantity. Next, in step 1204, the probe wirelessly transmits the data to the auxiliary device, and the auxiliary device receives the data from the probe. Next, in step 1205, the auxiliary device transmits the data (after optional processing) to a host device. Next, in step 1206, the host device receives the data from the auxiliary device and evaluates the physical stimulus based on the signal received by the auxiliary device. Moreover, in step 1207, optionally at least one of the host device and the auxiliary device may determine a control command for the wireless probe, based on information generated and transmitted by the probe and received by the auxiliary device. That information may comprise e.g. status information of the probe or another measurement such as a field strength or an amount of energy stored within the probe. Alternatively, the control command may be a command for the auxiliary device (for example, to provide more or less wireless power to the probe). Yet alternatively, the control command may be a command for the field generator of a navigation system to adjust a field strength. Yet alternatively, the control command may be generated based on information generated by the auxiliary device (for example a signal strength of data communication with the probe) in addition to (or alternatively to) information generated by the probe.

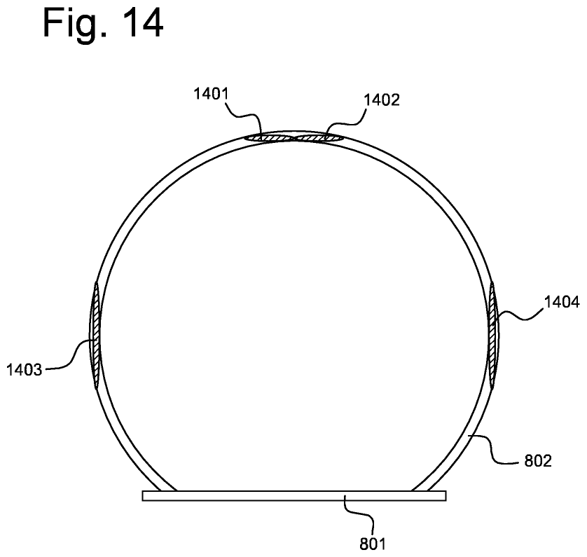

FIG. 14 shows a cross section of a rigid board 801 and a flexible board 802 of a probe. Only the rigid board 810, flexible board 802, light emitters 1401, 1402, and light detectors 1403, 1404 are shown in FIG. 14. Other components of the probe are omitted. In this example embodiment, two light emitters 1401 and 1402, for example light emitting diodes (LEDs) are fixed close to each other near a first position on the flexible board 802. The first light emitter 1401 is configured to emit light in a first frequency, and the second light emitter 1402 is configured to emit light at a second frequency, which is different from the first frequency. At a first detector position on the flexible board 802, a first photodetector 1403 is fixed. At another detector position on the flexible board 802, a second photodetector 1404 is fixed. The configuration allows to detect how the tissue around the probe changes the light emitted by the light emitters 1401, 1402. For example, only one light emitter emits light at any given time, so that it is clear from which light emitter the detected light originates. By analysing the detected vs. the emitted light (in conjunction with the relevant frequency of each light emitter), it is possible to characterize the tissue. Having two photodetectors 1403, 1404 instead of just one may improve the accuracy of the measurement.

In general, it may be advantageous to provide a configuration of the probe such that the direct line of sight between the light emitter(s) and the light detector(s) is obstructed by the material of the probe. This way, the only detected light is light diffracted or diffusing through the tissue in which the device is implanted. In other words, no direct light rays from the light emitter can reach the light detector. For example, referring to FIG. 14, the light emitter 1401, 1402 and the light detector 1403, 1404 are provided at least 90 degrees apart when viewing the cross section of the probe, as illustrated by means of an example in FIG. 14.

It will be understood that the techniques disclosed herein with respect to an implantable probe may be applied to any wireless probe. In alternative embodiments, the probes may be wireless probes that are not implantable. For example, wireless probes that may be attached to objects using an adhesive or a connector, or wireless probes that are built into another object, such as an instrument.

Some or all aspects of the invention may be suitable for being implemented in form of software, in particular a computer program product. The computer program product may comprise a computer program stored on a non-transitory computer-readable media. Also, the computer program may be represented by a signal, such as an optic signal or an electro-magnetic signal, carried by a transmission medium such as an optic fiber cable or the air. The computer program may partly or entirely have the form of source code, object code, or pseudo code, suitable for being executed by a computer system. For example, the code may be executable by one or more processors.

The examples and embodiments described herein serve to illustrate rather than limit the invention. The person skilled in the art will be able to design alternative embodiments without departing from the spirit and scope of the present disclosure, as defined by the appended claims and their equivalents. Reference signs placed in parentheses in the claims shall not be interpreted to limit the scope of the claims. Items described as separate entities in the claims or the description may be implemented as a single hardware or software item combining the features of the items described.

Related subject-matter is disclosed in the following clauses.

1. A tumor sensor system comprising an implantable probe, the probe comprising
at least one sensor configured to generate an electric signal in response to a physical stimulus;
at least one antenna for data communication; and
a processing circuit for generating data based on the signal generated by the sensor and transmitting the data via the at least one antenna.
   2. The system of clause 1, wherein the probe further comprises an energy harvesting coil for harvesting energy from an electromagnetic signal.
   3. The system of clause 2, wherein the energy harvesting coil of the probe is among the at least one antenna.
   4. The system of any preceding clause, wherein an antenna among the at least one antenna of the probe comprises a coil configured to communicate by means of at least one of near-field communication, far-field communication, electromagnetic resonance, or inductance.
   5. The system of any preceding clause, wherein the at least one sensor comprises a position sensor configured to sense a signal from a position tracking system wherein the sensed signal is indicative of a position of the probe in a space.
   6. The system of clause 5, wherein the position sensor comprises at least one electromagnetic sensor configured to cooperate with an external electromagnetic field generator, wherein the electromagnetic sensor is configured to detect a property of the electromagnetic field and send it as an electric signal to the processing circuit.
   7. The system of any one of clauses 5 to 6, wherein the position sensor of the probe comprises at least two electromagnetic sensors, which are oriented in at least two angularly distinct directions.
   8. The system of any preceding clause, wherein the at least one sensor comprises at least one of:
an ionizing radiation dose detector;
a peak ionizing radiation dose detector;
a cumulative ionizing radiation dose detector;
a sensor configured to detect a property of a tissue surrounding the probe;
at least two electrodes to detect an impedance of a tissue external to the probe; and
at least one photodetector with optional light emitters.
   9. The system of any preceding clause, wherein the probe comprises
a rigid circuit; and
a flex circuit following a circumference of the implantable transponder,
wherein the components of the probe are arranged on the rigid circuit and the flex circuit.
   10. The system of any preceding clause,
further comprising a power supply coil external to the probe, wherein the power supply coil is configured to transmit a wireless energy-containing electromagnetic signal to the probe.
   11. The system of any preceding clause, wherein a length of the probe is at most 2 centimeters, and/or wherein a width of the probe is at most 3 millimeters.
   12. The system of any preceding clause, wherein the system further comprises an auxiliary device, the auxiliary device comprising
an antenna configured to receive the data from the probe; and
a transmitter configured to transmit data based on the received data to a host.
   13. A tumor sensor system comprising an auxiliary device, the auxiliary device comprising
an antenna configured to receive data from an implantable probe; and
a transmitter configured to transmit data based on the received data to a host.
   14. The system of clause 12 or 13, wherein the auxiliary device comprises a coil for wirelessly supplying power to the probe by transmitting an energy-containing electromagnetic signal to the probe.
   15. The system of any one of clauses 12 to 14, wherein the auxiliary device is a handheld device with a housing encapsulating the antenna of the auxiliary device and the transmitter of the auxiliary device.
   16. The system of any one of clauses 12 to 15, wherein the auxiliary device comprises a wired power supply configured to receive electric energy through a wire.
   17. The system of any one of clauses 12 to 15, wherein the auxiliary device comprises fixation means for fixing the auxiliary device to a patient or to an interventional patient support.
   18. The system of any one of clauses 12 to 17, wherein the auxiliary device is suitable for being temporarily placed in a human or animal living being during a treatment session.
   19. The system of any one of clauses 12 to 16, wherein the auxiliary device comprises a surgical instrument, such as a hand-held surgical instrument.
   20. The system of any one of clauses 12 to 19, wherein the auxiliary device comprises a position sensor.
   21. The system of any one of clauses 12 to 20, further comprising a robot arm configured to hold and move the auxiliary device during surgery.
   22. The system of any preceding clause, wherein the probe and/or the auxiliary device is detectable by X-ray, ultrasound, or magnetic resonance imaging, MRI.
   23. The system of any preceding clause, further comprising a feedback control loop, wherein at least one of:
the auxiliary device is configured to control a power emitted by the coil for transmitting the energy-containing EM signal in dependence on a signal sent from the probe to the auxiliary device;
the electromagnetic field generated by the field generator depends on a signal sent from the probe to the auxiliary device; or
the processing circuit of the probe is configured to set an operating parameter of the probe based on a signal transmitted from the auxiliary device to the probe.
   24. A method performed by an implantable probe in a tumor sensor system, the method comprising
generating an electric signal in response to a physical stimulus by at least one sensor;
generating, by a processing circuit, data based on the signal generated by the sensor; and
transmitting the data via an antenna.
   25. A method performed by an auxiliary device in a tumor sensor system, the method comprising
receiving data from an implantable probe by an antenna; and
transmitting data based on the received data by a transmitter to a host.

1. A tumor sensor system comprising an implantable probe, an auxiliary device, and a host device,
wherein the implantable probe comprises:
at least one sensor configured to generate an electric signal in response to a physical stimulus;
at least one antenna for data communication by means of inductive communication;
an energy harvesting coil for harvesting energy from an energy-containing electromagnetic signal; and
a processing circuit configured to generate data based on the signal generated by the sensor and transmitting the data, using digital communication, via the at least one antenna to the auxiliary device,
wherein the auxiliary device comprises:
an antenna configured to receive the data from the implantable probe by means of the inductive communication;
a transmitter configured to transmit data based on the received data to the host device; and
a coil for wirelessly supplying power to the probe by transmitting energy-containing electromagnetic signal to the probe; and
wherein the host device is configured to process or visualize data, and
wherein the auxiliary device is movable with respect to the host device and the implantable probe.

2. (canceled)

3. (canceled)

4. The system of claim 1, wherein the system is configured to actively monitor a resonance state of at least one coil of the probe or of the auxiliary device, and actively control an operation of the probe or the auxiliary device based on the monitored resonance state.

5. (canceled)

6. (canceled)

7. (canceled)

8. The system of claim 1, wherein the at least one sensor comprises at least one of:
an ionizing radiation dose detector;
a peak ionizing radiation dose detector;
a cumulative ionizing radiation dose detector;
a sensor configured to detect a property of a tissue surrounding the probe;
at least two electrodes to detect an impedance of a tissue external to the probe; and
at least one photodetector with optional light emitters.

9. The system of claim 1, wherein the probe comprises
a rigid circuit; and
a flex circuit following a circumference of the implantable probe,
wherein the components of the probe are arranged on the rigid circuit and the flex circuit.

10. The system of claim 9, wherein two opposite edges of the flex circuit are attached to the rigid PCB.

11. (canceled)

12. (canceled)

13. (canceled)

14. The system of claim 1, wherein at east one of:
the auxiliary device is a handheld device with a housing encapsulating the antenna of the auxiliary device and the transmitter of the auxiliary device;
the probe and/or the auxiliary device is detectable by X-ray, ultrasound, or magnetic resonance imaging, MRI; or
the system comprises a surgical instrument comprising a further probe comprising a further position sensor.

15. The system of claim 1, wherein the auxiliary device comprises a wired power supply configured to receive electric energy through a wire.

16. The system of claim 1, wherein the auxiliary device comprises fixation means for fixing the auxiliary device to a patient or to an interventional patient support.

17. The system of claim 1, wherein the auxiliary device is suitable for being temporarily placed in a human or animal living being during a treatment session.

18. The system of claim 1, wherein the auxiliary device comprises a surgical instrument or a position sensor.

19. (canceled)

20. The system of claim 1, further comprising a robot arm configured to hold and move the auxiliary device during surgery.

21. (canceled)

22. The system of claim 1, further comprising a feedback control loop, wherein at least one of:
the auxiliary device is configured to control a power emitted by the coil for transmitting the energy-containing EM signal in dependence on a signal sent from the probe to the auxiliary device;
the electromagnetic field generated by the field generator depends on a signal sent from the probe to the auxiliary device; or
the processing circuit of the probe is configured to set an operating parameter of the probe based on a signal transmitted from the auxiliary device to the probe.

23. (canceled)

24. A method performed by an implantable probe, and an auxiliary device, and a host device in a tumor sensor system, the method comprising
generating, by a sensor or the implantable probe, an electric signal in response to a physical stimulus;
generating, by a processing circuit of the implantable probe, data based on the signal generated by the sensor;
transmitting, by the processing circuit of the implantable probe, the data via an antenna of the implantable probe to the auxiliary device by means of inductive communication;
harvesting, by an energy harvesting coil of the implantable probe, energy from an energy-containing electromagnetic signal;
receiving, by an antenna of the auxiliary device, the data from the probe, by means of inductive communication;
transmitting, by a transmitter of the auxiliary device, data based on the received data to the host device;
wirelessly, by a coil of the auxiliary device, supplying power to the probe by transmitting the energy-containing electromagnetic signal to the probe; and
processing or visualizing the data, by the host device,
wherein the auxiliary device is movable with respect to the host device and the implantable probe.

25. The system of claim 1, wherein the auxiliary device further comprises a housing with the antenna, the transmitter, and the coil inside the housing.

26. The system of claim 1, wherein the system is configured to maintain the energy harvesting coil of the implantable probe at resonance to improve energy transfer.

27. The system of claim 26, wherein the auxiliary device is configured to change transmission frequency according to a signal sent from the implantable probe.

28. The system of claim 26, wherein the processing circuit of the implantable probe is configured to detect the resonance condition of the energy harvester coil and modify an impedance of a circuit based on the detected resonance condition.

29. The system of claim 1, wherein the implantable probe further comprises a power management module to control at least one of energy harvesting, energy storage, and energy consumption.

30. The system of claim 1, wherein the at least one sensor comprises a position sensor configured to sense a signal from a position tracking system, wherein the sensed signal is indicative of a position of the implantable probe in space.

31. The system of claim 30, wherein the position sensor comprises at least one electromagnetic sensor configured to cooperate with an external electromagnetic field generator, wherein the electromagnetic sensor is configured to detect a property of the electromagnetic field and send it as an electric signal to the processing circuit, wherein the sensed signal of the position sensor is indicative of the position of the implantable probe with respect to the external electromagnetic field generator,
wherein the auxiliary device is further movable with respect to the external electromagnetic field generator, and the processing circuit of the implantable probe is configured to set an operating parameter based on a signal transmitted from the auxiliary device to the implantable probe.
